(19) United States
(12) Patent Application Publication
(10) Pub. No.: US 2016/0279041 A1
(43) Pub. Date: Sep. 29, 2016

(54) Cosmetic Products

(71) Applicant: BASF SE, Ludwigshafen (DE)

(72) Inventors: Matthias Hloucha, Koeln (DE); Esther Kuesters, Erkrath (DE); Jasmin Schorb, Monheim (DE); Claus Nieendick, Krefeld (DE); Mirella Winzek, Titz (DE); Stephanie Seidler, Duesseldorf (DE)

(73) Assignee: BASF SE, Ludwigshafen (DE)

(21) Appl. No.: 14/914,681

(22) PCT Filed: Aug. 19, 2014
(86) PCT No.: PCT/EP2014/067610

§ 371 Date: Feb. 26, 2016

(30) Foreign Application Priority Data
Aug. 29, 2013 (EP) 13182190.2

(51) Int. Cl. A61K 8/36; A61K 8/86; A61Q 19/10; A61K 8/97; A61K 8/37; A61K 8/33; A61K 8/31

(52) U.S. Cl. CPC A61K 8/36; A61K 8/33; A61K 8/86; A61K 8/31; A61K 8/97; A61K 8/37; A61Q 19/10; A61K 2800/48; A61K 2800/596

(57) ABSTRACT

The invention relates to oil concentrates with liquid anionic surfactants selected from the group of the alk(en)yl polyglycol ether citrates and with nonionic surfactants, suitable for producing cosmetic cleansing compositions.

### FIELD OF THE INVENTION

The invention is situated within the field of oil-containing cosmetic compositions, and pertains to oil concentrates suitable for producing cosmetic compositions of this kind, and also to the cosmetic compositions and their production.

### PRIOR ART

Cosmetic compositions are used for the cleansing and/or care of hair and skin. In the cleansing of hair or skin as well, ideally, the losses of lipids and water that are caused by washing are kept as low as possible, in order as far as possible to retain softness and smoothness in the hair and/or skin. As far as possible, therefore, cosmetic compositions such as shower gels, bath foams, or hair shampoos are required to fulfill care requirements as well.

WO 2008/155075 describes cosmetic preparations which as well as unalkoxylated surfactants comprise a microemulsion composed of alkylpolyglycoside, glycerol monoester, an oily substance, and water. These cosmetic preparations are suitable as hair shampoos with conditioning action, since they improve the deposition of silicone oils and introduce oils featuring softness and moistness into hair shampoos, in relatively large amounts.

WO 2011/116881 in turn discloses cosmetic cleansing compositions which comprise microemulsions of alkyl(oligo)glycoside, co-surfactant, a non-water-soluble organic oil component, and also a wax and water. Barring alkoxylated compounds, these microemulsions can comprise a large number of oily substances such as C₆-C₁₀ fatty acid triglycerides, vegetable oils, or hydrocarbons, as well as the wax. These waxy microemulsions allow significantly higher deposition of lipid components on the skin, thereby eliminating the adverse effects of detersive substances, such as drying of the skin, without lessening the positive cleansing activity of the surfactants.

Known from German laid-open specification DE 10 2007 063 134 is a method for producing oil-in-water emulsions, whereby an emulsifier concentrate is brought into contact with an oil phase in a laminar flow field. This method does allow high flexibility in terms of the nature of emulsifiers and of the oil phase, but is tied to the laminar flow field.

The European patent application discloses liquid shower oils which comprise essentially oil and also oil-soluble liquid surfactants preferably selected from the group of fatty alcohol ether sulfates and/or fatty alcohol ether phosphates. Shower oils of this kind do not exhibit a voluminous foam.

The desire of consumers, however, is for cleansing cosmetic compositions such as shower gels and hair shampoos that exhibit an attractive, stable, fine foam, despite comprising waxes and oil components that provide protection for the skin and have a foam-damping effect. For many consumers, indeed, large volumes of foam are synonymous with effective cleansing, and small quantities of foam lead the consumer to use unnecessarily large quantities of shower gels or hair shampoos, respectively.

It was an object of the present invention to provide oil-containing cosmetic compositions which as well as the cleansing effect enable very good care performance for skin and hair and which in spite of foam-damping oil components exhibit a voluminous foam. These oil-containing cosmetic compositions are intended, moreover, to be able to be produced in a simple way, as far as possible in a cold operation.

Surprisingly it has now been found that oil-containing cosmetic compositions of this kind are obtained when using oil concentrates which comprise alk(en)yl polyglycol ether citrates as liquid anionic surfactants, nonionic surfactants, and a selected oil component, having particular polarities. These oil concentrates, surprisingly, exhibit near-spontaneous self-emulsification with water, and lead to fine O/W emulsions. Using an oil concentrate of this kind allows the production, simply and stably, of cosmetic compositions with large amounts of oil.

The cosmetic compositions of the invention that are obtained are transparent, exhibit high cleansing performance and good lipid deposition, display good sensory tactile qualities and excellent foam behavior—that is, a good and long-lasting foam. The production method for the oil-containing cosmetic compositions is very simple and untroublesome, and can take place in a cold operation, to give the cosmetic compositions in the form of fine and stable emulsions.

### DESCRIPTION OF THE INVENTION

A subject of the present invention are oil concentrates for cosmetic compositions, comprising
(a) at least one anionic surfactant which is liquid at room temperature and is selected from the group of the alk(en)yl polyglycol ether citrates, and
(b) at least one nonionic surfactant, and
(c) an oil component with
(c1) a polar oil having a polarity of between 5 and 30 mN/m and optionally
(c2) an apolar oil having a polarity of above 35 mN/m, the (c1):(c1+c2) mixing ratio being in the range from 0.1 to 1,
(d) and also, optionally, thickeners.

Oil concentrates in the sense of the invention are mixtures which comprise oil in high quantities, preferably in quantities of 30 to 60 wt %—based on oil concentrate.

In the sense of the invention, the oil concentrates comprise anionic alk(en)yl polyglycol ether citrates which are liquid at room temperature as surfactants (a), meaning that they are present in liquid form in the range from about 10 to 30° C. In the sense of the invention, no dilutions of the anionic surfactants are meant, such as aqueous solutions, aqueous gels, or oily suspensions; instead, the anionic compounds (a) as far as possible have an active-substance content of virtually 100 wt %, preferably having active-substance contents of between 90 to 100, preferably 95 to 100 wt %.

Anionic surfactants (a) in the sense of the invention are not only surfactants which are already present in anionic form, as salt, but also surfactants which are converted into the anionic form (undergo dissociation) only when used in aqueous solution. In other words, the anionic surfactants comprise at least one dissociated group or are converted into such a group in aqueous solution, optionally with addition of pH modifiers or pH regulators. In the sense of the present invention, therefore, the term “anionic surfactants” always also comprehends the salts of the alk(en)yl polyglycol ether citrates.

The alk(en)yl polyglycol ether citrates and salts thereof may be mono-, di- and/or triesters of citric acid and alkoxylated alcohols conforming to the formula (I):

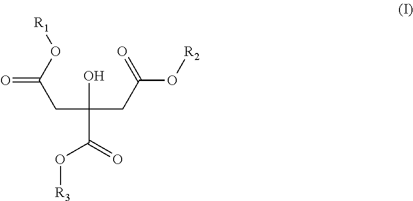

in which
R₁, R₂, and R₃ independently of one another are hydrogen or the radical of the formula

R₄(OCH₂CHR₅)ₙ (II)

in which
R₄ is a linear or branched alkyl and/or alkenyl radical having 6 to 22 carbon atoms,
R₅ is hydrogen or methyl radical, and
n is a number from 1 to 20, subject to the condition that at least one of the radicals, R₁, R₂, or R₃, is other than hydrogen.

As salts of the alk(en)yl polyglycol ether citrates, the corresponding carboxyl groups are present in dissociated form.

Preferred in the sense of the invention are alk(en)yl polyglycol ether citrates of the formula (I) in which R₁, R₂, or R₃ is the radical of the formula (II) and R₄ is a linear or branched alkyl and/or alkenyl radical having 6 to 22 carbon atoms, R₅ is hydrogen, and n is a number from 1 to 20, preferably 5 to 10.

Typical examples of the alkoxylated alcohol moiety of the esters conforming to formula (II) are adducts of on average 1 to 20 mol, preferably 5 to 10 mol, of ethylene oxide with caproyl alcohol, caprylyl alcohol, 2-ethylhexyl alcohol, capryl alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, and brassidyl alcohol, and technical mixtures thereof.

Particularly preferred are alk(en)yl polyglycol ether citrates of the formula (I) in which R₁, R₂, or R₃ is the radical of the formula (II) and R₄ is a linear alkyl radical having 12 to 14 carbon atoms, R₅ is hydrogen, and n is a number from 5 to 10.

Especially preferred are technical mixtures of alk(en)yl polyglycol ether citrates of the formula (I) in which R₁, R₂, or R₃ are the radical of the formula (II), subject to the condition that in amounts of at least 50 wt %, preferably 70 wt %, more particularly 75 to 80 wt %—based on alk(en)yl polyglycol ether citrates—only one of the radicals, R₁, R₂, or R₃, is other than hydrogen.

Outstandingly suitable are alkyl polyalkylene glycol ether citrates based on adducts of 5 to 10, more particularly approximately 7, mol of ethylene oxide with technical C₁₂-C₁₈, more particularly C₁₂-C₁₄, fatty alcohol fractions. Especially preferred are the polyethylene glycol ethers of lauryl alcohol, Laureth-7 citrates, which are available for example under the name Plantapon® LC₇ (BASF & Personal Care & Nutrition GmbH).

If desired, the oil concentrates of the invention may comprise not only the alk(en)yl polyglycol ether citrates but also further anionic surfactants, which either themselves are liquid at room temperature or then advantageously are in solution in the oil concentrate. Examples of further anionic surfactants, in each case in the form of their salts, are ethercarboxylic acids, acylsarcosides having 8 to 24 C atoms in the acyl group, acyltaurides having 8 to 24 C atoms in the acyl group, acylisethionates having 8 to 24 C atoms in the acyl group, sulfosuccinic monoalkyl and dialkyl esters having 8 to 24 C atoms in the alkyl group, and sulfosuccinic monoalkyl polyoxyethyl esters having 8 to 24 C atoms in the alkyl group and 1 to 6 oxyethyl groups, linear alkanesulfonates having 8 to 24 C atoms, linear alpha-olefinsulfonates having 8 to 24 C atoms, alpha-sulfo fatty acid methyl esters of fatty acids having 8 to 30 C atoms, alkyl sulfates, alkyl polyglycol ether sulfates, esters of tartaric acid and citric acid, alkyl and/or alkenyl ether phosphates, sulfated fatty acid alkylene glycol esters, monoglyceride sulfates and monoglyceride ether sulfates, and also condensation products of C₈-C₃₀ fatty alcohols with protein hydrolysates and/or amino acids and derivatives thereof, referred to as protein fatty acid condensates, e.g., Lamepon®, Gluadin®, Hostapon® KCG, or Amisoft®.

The anionic surfactants (a) may be present in amounts of 20 to 40 wt %, preferably 25 to 35 wt %, and more particularly 27 to 32 wt %—based on oil concentrate.

In particular, alk(en)yl polyglycol ether citrates, preferably of the formula (I), are present exclusively as anionic surfactant (a) in the oil concentrates of the invention.

A further ingredient of the oil concentrates of the invention are (b) nonionic surfactants. Examples of nonionic surfactants (b) are:

Adducts of 2 to 30 mol of ethylene oxide and/or 0 to 5 mol of propylene oxide with linear fatty alcohols having 8 to 22 C atoms, with fatty acids having 12 to 22 C atoms, with alkylphenols having 8 to 15 C atoms in the alkyl group, and also alkylamines having 8 to 22 carbon atoms in the alkyl radical;

alkyl- and/or alkenyloligoglycosides having 8 to 22 carbon atoms in the alk(en)yl radical and their ethoxylated analogs;

adducts of 1 to 15 mol of ethylene oxide with castor oil and/or hydrogenated castor oil;

adducts of 15 to 60 mol of ethylene oxide with castor oil and/or hydrogenated castor oil;

partial esters of glycerol and/or sorbitan with unsaturated, linear or saturated, branched fatty acids having 12 to 22 carbon atoms and/or hydroxycarboxylic acids having 3 to 18 carbon atoms, and also the adducts thereof with 1 to 30 mol of ethylene oxide;

partial esters of polyglycerol (average degree of self-condensation 2 to 8), polyethylene glycol (molecular weight 400 to 5000), trimethylolpropane, sugar alcohols (e.g., sorbitol), alkylglycosides (e.g., methylglucoside, butylglucoside, laurylglucoside), and also polyglycosides (e.g., cellulose) with saturated and/or unsaturated, linear or branched fatty acids having 12 to 22 carbon atoms and/or hydroxycarboxylic acids having 3 to 18 carbon atoms, and also the adducts thereof with 1 to 30 mol of ethylene oxide.

Further examples of nonionic surfactants are:

mixed esters of pentaerythritol, fatty acids, citric acid, and fatty alcohol and/or mixed esters of fatty acids having 6 to 22 carbon atoms, methylglucose and polyols, preferably glycerol or polyglycerol; mono-, di-, and trialkyl phosphates and also mono-, di- and/or tri-PEG-alkyl phosphates and their salts; lanolin alcohols; polysiloxane-polyalkyl polyether copolymers and corresponding derivatives; block copolymers, for example, polyethylene glycol 30 dipolyhydroxystearate; polymer emulsifiers, polyalkylene glycols and/or glycerol carbonate.

Preferred nonionic surfactants (b) are adducts of ethylene oxide and/or propylene oxide with fatty alcohols, fatty acids, alkylphenols, or with castor oil, partial esters of glycerol, partial esters of sorbitan, alkyl- and/or alkenyloligoglycosides and/or esters of polyglycerols.

The adducts of ethylene oxide and/or of propylene oxide with fatty alcohols, fatty acids, alkylphenols, or with castor oil constitute known products which are available commercially. These are mixtures of homologs whose average degree of alkoxylation corresponds to the ratio of the amounts-of-substance of ethylene oxide and/or propylene oxide and substrate with which the addition reaction is carried out.

Typical examples of partial esters of glycerol are hydroxystearic monoglyceride, hydroxystearic diglyceride, isostearic monoglyceride, isostearic diglyceride, oleic monoglyceride, oleic diglyceride, ricinoleic monoglyceride, ricinoleic diglyceride, linoleic monoglyceride, linoleic diglyceride, linolenic monoglyceride, linolenic diglyceride, erucic monoglyceride, erucic diglyceride, tartaric monoglyceride, tartaric diglyceride, citric monoglyceride, citric diglyceride, malic monoglyceride, malic diglyceride, and their technical mixtures which to a minor degree may still comprise small amounts of triglyceride from the preparation process. Likewise suitable are adducts of 1 to 30, preferably 5 to 10, mol of ethylene oxide with the stated partial glycerides.

Sorbitan partial esters contemplated include sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate, and also technical mixtures thereof. Likewise suitable are adducts of 1 to 30, preferably 5 to 10, mol of ethylene oxide with the stated sorbitan esters.

Also suitable nonionic surfactants are alkyl- and/or alkenyloligoglycosides, which are particularly skin-friendly. The compounds and their preparation are known from the prior art, being prepared more particularly by reaction of glucose or oligosaccharides with primary alcohols having 8 to 22 C atoms, preferably 12 to 22, and more preferably 12 to 18 C atoms. With respect to the glycoside radical it is the case that not only monoglycosides, in which one cyclic sugar radical is bonded glycosidically to the fatty alcohol, but also oligomeric glycosides, with a degree of oligomerization of up to preferably about 8, are suitable. This degree of oligomerization is a statistical average based on a homolog distribution customary for technical products of this kind. Products available under the Plantacare® designation comprise a glucosidically bonded C₈-C₁₆ alkyl group on an oligoglucoside radical whose average degree of oligomerization is 1 to 2. Also suitable as nonionic surfactants are the glucamine-derived acylglucamides.

Especially preferred as nonionic surfactants in the oil concentrates of the invention are (b) esters of polyglycerols. The acid component of these polyglycerol esters may derive from straight-chain, branched, saturated and/or unsaturated carboxylic acids, optionally with functional groups such as hydroxyl groups. With particular preference the acid component comprises isostearic acid and/or poly-12-hydroxystearic acid.

According to one embodiment, the polyglyceryl-3 diisostearate which is marketed by BASF Personal Care and Nutrition GmbH under the Lameform®TGI designation proved to be highly suitable as nonionic surfactant (b) for producing oil concentrates which are readily self-emulsifiable in water.

According to one particularly preferred further embodiment, esters of poly-12-hydroxystearic acid with polyglycerols, and more particularly those of polyglycerols with the following homolog distribution, proved advantageous (preferred amounts are indicated in parentheses):

Glycerols: 5 to 35 (15 to 30) wt %

Diglycerols: 15 to 40 (20 to 32) wt-%

Triglycerols 10 to 35 (15 to 25) wt %

Tetraglycerols: 5 to 20 (8 to 15) wt %

Pentaglycerols: 2 to 10 (3 to 8) wt %

Oligoglycerols: ad 100 wt %

Especially suitable is the poly(12-hydroxystearic acid) polyglycerol ester that is marketed for example by BASF Personal Care and Nutrition GmbH under the Dehymuls® PGPH designation.

In the oil concentrates of the invention, the nonionic surfactants (b) are present in amounts of 10 to 30 wt %, preferably 15 to 25 wt %.

The oil concentrates of the invention further comprise an oil component. The oil component necessarily comprises a polar oil (c1), either as sole oil component or in a mixture with an apolar oil (c2).

In other words: the oil concentrates of the invention comprise the oil component (c) with a polar oil (c1) having a polarity of between 5 and 30 mN/m and optionally with an apolar oil (c2) having a polarity above 35 mN/m, the (c1):(c1+c2) mixing ratio being in the range from 0.1 to 1.

The mixing ratio means the ratio of the mass of (c1) relative to the mass of (c1+c2). Where there is no apolar oil present (c2=0), the (c1):(c1+c2) mixing ratio corresponds to the value of 1, i.e., only the polar oil (c1) is present.

The term “oil” in the present description stands for a water-insoluble, organic, natural, or synthetic, cosmetically suitable oil which preferably has a liquid or viscous consistency at 23° C. “Water-insoluble” in this context refers to a water-solubility of the oil of not more than 2 wt % at 20° C.

In the sense of the present invention, the oil component (c) comprises at least one polar oil (c1) having a polarity of between 5 and 30 mN/m, preferably 15 to 28 mN/m.

It has proven advantageous for the oil component (c) to comprise a further, apolar oil (c2) having a polarity above 35 mN/m, preferably between 35 and 60 mN/m.

In one preferred embodiment the oil component (c) has a (c1):(c1+c2) mixing ratio of 0.3 to 0.7, preferably of 0.4 to 0.6, and more particularly of about 0.45 to 0.55.

The polarity of an oil is defined as the polarity index (interfacial tension) of the oil with respect to water. The polarity was determined using a ring tensiometer (e.g., Krüss K 10), which measures the interfacial tension in mN/m in analogy to the ASTM method D971-99a (2004).

In the sense of the present invention, the oils having a polarity above 35 mN/m are referred to as apolar, and those having a polarity of between 5 and 30 mN/m as polar.

Suitable polar oils (c1) are, for example, Guerbet alcohols based on fatty alcohols having 6 to 18, preferably 8 to 10, carbon atoms (e.g., Eutanol® G), esters of linear C₆-C₂₂ fatty acids with linear or branched C₆-C₂₂ fatty alcohols and/or esters of branched C₆-C₁₃ carboxylic acids with linear or branched C₆-C₂₂ fatty alcohols, such as myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, oleyl myristate, oleyl palmitate, oleyl stearate, oleyl isostearate, oleyl oleate, oleyl behenate, oleyl erucate (Cetiol J600®), behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate, and erucyl erucate under consideration. Suitable as well are esters of linear C₆-C₂₂ fatty acids with branched alcohols, more particularly 2-ethylhexanol, esters of C₃-C₃₈ alkylhydroxycarboxylic acids with linear or branched C₆-C₂₂ fatty alcohols, especially diethylhexyl malate, esters of linear and/or branched fatty acids with polyhydric alcohols (such as propylene glycol, dimer diol, or trimer triol, for example) and/or Guerbet alcohols, triglycerides based on C₆-C₁₀ fatty acids, liquid mono-/di-triglyceride mixtures based on C₆-C₁₈ fatty acids, esters of C₆-C₂₂ fatty alcohols and/or Guerbet alcohols with aromatic carboxylic acids, especially benzoic acid, esters of C₂-C₁₂ dicarboxylic acids with linear or branched alcohols having 1 to 22 carbon atoms or polyols having 2 to 10 carbon atoms and 2 to 6 hydroxyl groups, vegetable oils, branched primary alcohols, substituted cyclohexanes, linear and branched C₆-C₂₂ fatty alcohol carbonates, such as Dicaprylyl Carbonate (Cetiol® CC), Guerbet carbonates based on fatty alcohols having 6 to 18, preferably 8 to 10, C atoms, esters of benzoic acid with linear and/or branched C₆-C₂₂ alcohols (e.g., Finsolv® TN), linear or branched, symmetric or asymmetric dialkyl ethers having 6 to 22 carbon atoms per alkyl group, such as Dicaprylyl Ether (Cetiol® OE), for example, and ring-opening products of epoxidized fatty acid esters with polyols (Hydagen® HSP, Sovermol® 750, Sovermol® 1102).

In accordance with one embodiment of the present invention, polar oils (c1) used comprise mono-, di-, and tri-fatty acid esters of glycerol, which may have preferably 6 to 24 and more particularly 8 to 18 hydrocarbon atoms and be saturated and/or unsaturated, these esters being obtained by chemical synthesis from a natural (plant or animal) source. Preferred among these are compounds which derive from plant sources, and more particularly glycerol esters of fatty acids having 8 and/or 10 carbon atoms, preferably di- and/or triglyceride esters of the kind obtainable under the trade name Myritol® 312 from BASF Personal Care & Nutrition GmbH (INCI name: Caprylic/Capric triglyceride).

In accordance with a further embodiment of the present invention, polar oils (c1) used are those vegetable oils that are widely known within the cosmetics industry, such as groundnut oil, castor oil, coconut oil, corn oil, olive oil, palm kernel oil, sunflower oil, soybean oil, rapeseed oil, almond oil, grapeseed oil, safflower oil, wheatgerm oil, evening primrose oil, macadamia nut oil, argan oil, avocado oil, and so on.

Especially suitable in the sense of the invention are oil concentrates with polar oils (c1) selected from the group consisting of olive oil, soybean oil, sunflower oil, almond oil, argan oil and/or avocado oil.

Apolar oils (c2) which can be used include the oils widely known within the cosmetics industry, preferably hydrocarbon-based oils such as aliphatic and/or aromatic oils which have preferably 8 to 32, more particularly 15 to 20, carbon atoms. Examples are squalane, squalene, liquid paraffins, isohexadecane, isoeicodecane, polyolefins such as polydecenes, hydrogenated polyisobutenes, dialkylcyclohexane, and mineral oil. Preference is given to liquid paraffins such as the low-viscosity paraffins (paraffinum perliquidum), which have a viscosity of 25 to 80 mPa·s, and/or the high-viscosity paraffins (paraffinum subliquidum), which as oily liquids have a viscosity of 110 to 230 mPa·s. Also preferred are the hydrogenated polyisobutenes which are available, for example, under the trade name Luvitol® Lite from BASF SE. Deserving of emphasis in the sense of the invention as apolar oils (c2) are liquid paraffins and/or hydrogenated polyisobutenes.

Used very successfully as oil component (c) in the sense of the present invention is a mixture of

(c1) vegetable oils and

(c2) apolar hydrocarbon-based oils, and more particularly a mixture of

(c1) vegetable oils and

(c2) liquid paraffins.

One preferred embodiment of the present invention uses as oil component (c) a mixture of mono-, di- and/or tri-fatty acid esters of glycerol with fatty acids having 8 to 10 carbon atoms as (c1) and liquid paraffin as (c2).

According to a second preferred embodiment, a mixture of sunflower oil as (c1) and hydrogenated polyisobutene as (c2) is used as oil component (c).

The table below lists the polarities of the typical oils usually utilized.

| Oil (CTFA Name) | Polarity Index [mN/m] |
|---|---|
| apolar | |
| Isoparaffin (C₁₂-C₁₄) | 53.0 |
| Squalan ® | 46.2 |
| Isohexadecane (ARLAMOL ® ND) | 43.8 |
| Mineral oil (Paraffin oil perliquidum) | 43.7 |
| Mineral oil (Paraffin oil subliquidum) | 38.3 |
| Polar | |
| Oleyl Erucate (Cetiol J600 ®) | 27.1 |
| Passiflora Incarnata Oil (Cegesoft ® PFO) | 27.2 |
| Rapeseed oil | 21.9 |
| Myritol ® 312 | 25.3 |
| Cetystearyl octanoate | 28.6 |
| Dimethicone (silicon oil 20 ct) | 26.6 |
| Isopropyl palmitate | 25.2 |
| Octyldodecanol | 24.8 |
| Dioctyl adipate (ARLAMOL ® DOA) | 24.5 |
| Isopropyl myristate | 24.2 |
| Octyl palmitate (2-ethylhexyl palmitate) | 23.1 |
| Hexamethyldisiloxane | 22.7 |
| Isopropyl stearate | 21.9 |
| Caprylic/capric triglycerides | 21.3 |
| Isopropyl isostearate | 21.2 |
| Jojoba oil | 20.8 |
| Cyclomethicone (ARLAMOL ® D4) | 20.6 |
| Groundnut oil | 20.5 |
| Almond oil | 20.3 |
| Sunflower oil | 19.3 |
| Decyl oleate | 18.7 |
| Avocado oil | 18.3 |
| Olive oil | 16.9 |
| Castor oil | 13.7 |
| Calendula oil | 11.1 |
| Wheatgerm oil | 8.3 |

Further suitable oils are described in DE 102004003436 A1, and are incorporated here as well:

| Oil (CTFA Name) | Polarity Index [mN/m] |
|---|---|
| apolar | |
| Hydrogenated polyisobutene (Luvitol Lite ®) | 44.7 |
| Isohexadecane | 43.8 |
| Mineral oil | 43.7 |
| Isoeicosane | 41.9 |
| Dioctylcyclohexane | 39.0 |
| Polar | |
| Dicaprylyl carbonate | 31.7 |
| Dicaprylyl ether | 30.9 |
| Dihexyl carbonate | 30.9 |
| Jojoba oil Gold | 26.2 |
| Octyl palmitate | 23.1 |
| Macadamia nut oil | 22.1 |
| Isopropyl stearate | 21.9 |
| Dibutyl adipate | 14.3 |
| Propylene glycol monoisostearate | 6.2 |
| Cocoglycerides | 5.1 |

Also particularly preferred in the sense of the invention is soybean oil, which, with a polarity index of 13.5 mN/m, can be said to be polar.

In the sense of the invention, the oil component (c) is present preferably in amounts of 30 to 60 wt %, more preferably of 40 to 55 wt %—based on oil concentrate.

In the sense of the invention it is also possible for the oil component (c) to comprise waxes, in which case the fraction thereof is preferably below 20, more particularly below 15 wt %—based on oil component.

Waxes in the sense of the invention are natural substances of animal or plant origin which at room temperature (21° C.) are firm but generally possess a certain deformability. Waxes are insoluble in water but soluble in oils and capable of forming water-repellent films.

Typical examples of waxes in the sense of the present teaching are natural waxes, such as, for example, Shorea stenoptera butter (cegesoft SH), shea butter, candelilla wax, carnauba wax, japan wax, esparto grass wax, cork wax, guaruma wax, ricegerm oil wax, sugarcane wax, ouricury wax, montan wax, beeswax, shellac wax, spermaceti, lanolin (wool wax), uropygial grease, ceresin, ozokerite (earth wax), petrolatum, paraffin waxes, microwaxes; chemically modified waxes (hard waxes), such as, for example, montan ester waxes, Sasol waxes, hydrogenated jojoba waxes, and also synthetic waxes, such as polyalkylene waxes and polyethylene glycol waxes, for example. Preference is given to the natural waxes, especially preferably shea butter (also shea fat, carité fat or galam butter), which is a natural solid fatty substance obtained from the plant Butyrospermum parkii, the African shea butter tree, and is available in commercial quantities. Its melting range is 35 to 42° C. Shea butter customarily comprises 89 to 98 wt % of triglycerides, glycerol partial esters, and free fatty acids, and also a 2 to 11 wt % content of unsuponifiable components, of which the most important are hydrocarbons (karitenes), triterpene alcohols, and sterols.

The oil concentrates of the invention may further comprise a thickener as further optional component d), this having advantageous consequences for the viscosities of the cosmetic compositions into which they are introduced.

Suitable thickeners d) are xanthan gum, guar guar, agar agar, alginates and Tyloses, carboxymethylcellulose and hydroxyethyl and hydroxypropylcellulose, and also relatively higher molecular mass polyethylene glycol monoesters and diesters of fatty acids, polyacrylates and hydrophobically modified polyacrylates, polyacrylamides, polymers, polyvinyl alcohol, and polyvinylpyrrolidone. Bentonites as well have proven particularly effective, these being a mixture of cyclopentasiloxane, disteardimonium hectorite, and propylene carbonate. Further contemplated are surfactants, such as, for example, ethoxylated fatty acid glyerides, ethoxylated and/or propoxylated esters of fatty acids with polyols such as, for example, pentaerythritol or trimethylolpropane, fatty alcohol ethoxylates with narrowed homolog distribution, or alkyloligoglucosides, and also electrolytes such as sodium chloride and ammonium chloride.

The oil concentrates preferably comprise as d) mixtures of ethoxylated and/or propoxylated polyol esters of trimethylolpropane and ethoxylated fatty alcohols, and more particularly a mixture of an ethoxylated and propoxylated trimethylol oleate and an ethoxylated lauryl alcohol which is available under the trade name Arlypon® TT from BASF Personal Care & Nutrition GmbH.

Especially suitable oil concentrates comprise
(a) alk(en)yl polyglycol ether citrates in amounts of 20 to 40 wt %, preferably 25 to 35 wt % and
(b) at least one nonionic surfactant in amounts of 10 to 30 wt %, preferably 15 to 25 wt %, and also the
(c) oil component in amounts of 30 to 60 wt %, preferably 40 to 55 wt %, and also, optionally,
(d) thickeners in amounts of 0 to 15 wt %.

One especially suitable oil concentrate according to the present invention consists of

25 to 35 wt % of alkyl polyglycol ether citrate, preferably Laureth-7 citrate

15 to 25 wt % of esters of polyglycerols, more particularly poly-12-hydroxystearic acid

40 to 55 wt % of oil component (c) with a mixture of mono-, di- and/or tri-fatty acid esters of glycerol with fatty acids having 8 to 10 carbon atoms as (c1) and liquid paraffin as (c2) in a (c1):(c1+c2) mixing ratio of 0.45 to 0.55.

Another especially suitable oil concentrate of the present invention consists of

25 to 35 wt % of alkyl polyglycol ether citrate, preferably Laureth-7 citrate

15 to 25 wt % of esters of polyglycerols, more particularly poly-12-hydroxystearic acid

35 to 50 wt % of oil component (c) with a mixture of sunflower oil as (c1) and hydrogenated polyisobutene as (c2) in a (c1):(c1+c2) mixing ratio of 0.45 to 0.55, and 5 to 15 wt % of thickeners, preferably a mixture of an ethoxylated and propoxylated trimethyl oleate and an ethoxylated lauryl alcohol.

On the laboratory scale, the oil concentrates of the invention can be prepared by simple manual stirring at room temperature or at slightly elevated temperatures, preferably below 60° C. With preference the oil component (c) is introduced initially and the liquid anionic surfactant alk(en)yl polyglycol ether citrates (a) and also the nonionic surfactant (b) and optionally the thickener (d) are stirred in. Additional water, which is not introduced in the form of aqueous formulations of the surfactants and/or thickeners, can be added in the last method step, but is undesirable in the sense of the invention. Instead the desire is for oil concentrates which comprise virtually no water, preferably 0 to 5 wt %, preferably below 3 wt %, and more particularly between 0 wt % and 2 wt %.

The oil concentrates of the invention are clear and have low viscosities. The oil concentrates of the invention exhibit very high stability in storage for several weeks.

The oil concentrates of the invention can be mixed unproblematically with water, the concentrates preferably being added to an initial charge of water at room temperature, where very fine O/W emulsions are formed spontaneously without additional mechanical energy input (such as stirring).

The oil concentrates of the invention can be introduced accordingly without problems into water-containing cosmetic compositions to give cosmetic compositions having a high oil fraction. Via the pathway of the oil concentrates of the invention, oils can be introduced into the cosmetic compositions without laborious, high-shear stiring as part of a cold operation, in other words without high temperatures. Furthermore, the oil-containing cosmetic compositions produced accordingly are transparent, very fine, with particle sizes below 1 μm, and storage-stable. An additional great advantage is that the oil-containing cosmetic compositions also exhibit good foam properties.

In the sense of the present invention, the oil concentrates of the invention are used preferably in cosmetic cleansing compositions, more particularly in hair shampoos, hair lotions, bath foams, shower products, shower gels, shower creams, and combined hair and shower shampoos.

A further subject of the present invention is a method for producing oil-containing cosmetic compositions in a cold operation, in other words at room temperature, preferably in the range from 20 to 25° C., where the oil concentrates of the invention are stirred together with B) anionic surfactants and C) nonionic surfactants and D) cosmetic additives, and E) water.

The oil concentrates of the invention here may be introduced into an initial charge of components B), C), D) and/or E). Either all of the components or only part of them may be introduced initially, and they may also be introduced initially in total or only proportionally. In accordance with one possibility, the oil concentrate of the invention can be introduced into an initial charge of water E), with the further components of the cosmetic compositions, B) and C) and D), being added to the resultant premix. It has proven advantageous if only proportions of the total amount of water E) are introduced in the initial charge, and the remaining amount of water is not added until after the other components have been introduced.

The introduction of the oil concentrates of the invention into the cosmetic compositions is preferably assisted by brief stirring round or stirring through.

The cosmetic compositions comprise not only

A) the oil concentrates of the invention but also as further components

B) anionic surfactants

C) further, co-surfactants, different from B),

D) cosmetic additives, and

E) water.

Cosmetic compositions which comprise the oil concentrates in preformulated form surprisingly display improved properties, particularly with respect to appearance and stability in storage.

Hence even after prolonged storage of the cosmetic compositions of the invention, there is no observed separation of the oil of the kind observed if oil is introduced into the cosmetic compositions directly, in other words without preformulation as a concentrate.

The cosmetic compositions preferably comprise

A) 0.5 to 20 wt % of oil concentrates of the invention

B) 5.0 to 20 wt % of anionic surfactants

C) 1 to 15 wt % of further, co-surfactants, different from B),

D) 0.1 to 10 wt % of cosmetic additives, and

E) water ad 100 wt %.

Anionic surfactants B) present in the cosmetic compositions may be those selected from the group consisting of soaps, alkylbenzenesulfonates, alkanesulfonates, olefinsulfonates, alkyl ether sulfonates, glycerol ether sulfonates, α-methyl ester sulfonates, sulfo-fatty acids, alkyl sulfates, fatty alcohol ether sulfates, glycerol ether sulfates, fatty acid ether sulfates, hydroxyl-mixed ether sulfates, monoglyceride (ether) sulfates, fatty acid amide (ether) sulfates, mono- and dialkylsulfosuccinates, mono- and dialkylsulfosuccinamates, sulfotriglycerides, amide soaps, ethercarboxylic acids and their salts, fatty acid isethionates, fatty acid sarcosinates, fatty acid taurides, N-acylamino acids, such as acyllactylates, acyltartrates, acylglutamates, and acylaspartates, for example, alkyloligoglucoside sulfates, protein fatty acid condensates (especially plant products based on wheat), and alkyl (ether) phosphates. Particularly preferred in this context are the fatty alcohol ether sulfates such as, for example, sodium lauryl ether sulfate or other compounds having comparable foam behavior. Alkyl ether sulfates (“fatty alcohol ether sulfates”) represent known anionic surfactants, which are produced industrially by SO₃ or chlorosulfonic acid (CSA) sulfation of fatty alcohol or oxo-process alcohol polyglycol ethers and subsequent neutralization. Contemplated in the sense of the invention are ether sulfates which conform to the formula (II), R²O—(CH₂CH₂O)ₘSO₃X (II), in which R² is a linear or branched alkyl and/or alkenyl radical having 6 to 22 carbon atoms, n stands for numbers from 1 to 10, and X is an alkali metal and/or alkaline earth metal, ammonium, alkylammonium, alkanolammonium, or glucammonium. Typical examples are the sulfates of adducts of on average 1 to 10 and more particularly 2 to 5 mol of ethylene oxide with caproyl alcohol, caprylyl alcohol, 2-ethylhexyl alcohol, capryl alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, and brassidyl alcohol, and also their technical mixtures in the form of their sodium and/or magnesium salts. These ether sulfates may have either a conventional or else a narrowed homolog distribution.

Particularly preferred is the use of ether sulfates based on adducts of on average 2 to 3 mol of ethylene oxide with technical C₁₂/₁₄ and/or C₁₂/₁₈ coconut fatty alcohol fractions in the form of their sodium and/or magnesium salts. Preferred anionic surfactants B) are from the group consisting of fatty alcohol ether sulfate, monoglyceride (ether) sulfate, mono- and dialkylsulfosuccinate, ethercarboxylic acids and/or salts thereof, acylglutamates, and alkyloligoglucoside sulfates.

With particular preference the cosmetic compositions comprise
A) 5.0 to 15 wt % of oil concentrates of the invention
B) 5.0 to 20 wt % of anionic surfactants from the group consisting of fatty alcohol ether sulfate, monoglyceride ether sulfate, mono- and dialkylsulfosuccinate, ethercarboxylic acids and/or salts thereof, acylglutamates, and alkyloligoglucoside sulfates
C) 1 to 10 wt % of further, co-surfactants, different from B),
D) 0.5 to 5 wt % of cosmetic additives, and
E) water ad 100 wt %.

Suitable co-surfactants C) different from B) are in principle nonionic, amphoteric and/or cationic surfactants.

Typical examples of nonionic surfactants have already been described for the oil concentrates as component b), referred to expressly here in relation to the disclosure. Suitable nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers and/or mixed formals, optionally partially oxidized alk(en)yloligoglycosides and/or glucoronic acid derivatives, fatty acid N-alkylglucamides, protein hydrolysates (especially plant products based on wheat), polyol fatty acid esters, sugar esters, sorbitan esters, polysorbates, alkyl- and/or alkenyloligoglycosides, and amine oxides. Where the nonionic surfactants comprise polyglycol ether chains, they may have a conventional or, preferably, a narrowed homolog distribution.

Preferred as further co-surfactants C) in the sense of the invention are amphoteric or zwitterionic surfactants.

Preferred accordingly are cosmetic compositions comprising
A) 5.0 to 15 wt % of oil concentrates of the invention
B) 5.0 to 20 wt % of anionic surfactants selected from the group consisting of fatty alcohol ether sulfate, monoglyceride ether sulfate, mono- and dialkylsulfosuccinate, ethercarboxylic acids and/or salts thereof, acylglutamates, and alkyloligoglucoside sulfates
C) 1 to 10 wt % of amphoteric and/or cationic surfactants
D) 0.5 to 5.0 wt % of cosmetic additives, and
E) water ad 100 wt %.

Typical examples of cationic surfactants are quaternary ammonium compounds, such as dimethyldistearylammonium chloride, for example, and ester quats, more particularly quaternized fatty acid trialkanolamine ester salts.

Typical examples of amphoteric or zwitterionic surfactants are alkylbetaines, alkylamidobetaines, aminopropionates, aminoglycinates, imidazolinium betaines and sulfobetaines, with cocamidopropylbetaine being particularly preferred. The stated surfactants are exclusively known compounds. Typical examples of particularly suitable mild surfactants, these being surfactants which are particularly skin-compatible, are fatty acid glucamides, alkylamidobetaines such as cocoamidopropylbetaine, amphoacetates such as sodium cocoamphoacetate, and/or protein fatty acid condensates, the latter preferably based on wheat proteins. Present as C) with particular preference is cocoamidopropylbetaine.

The cosmetic additives D) may be selected from the group consisting of hydrotropes such as glycerol, preservatives, citric acid, phenoxyethanol, UV light protection filters, antioxidants, active biogenic ingredients, fragrance, dyes, biocides, defoamers, and pH regulators.

When using oil concentrates based on alkyl polyalkylene glycol ether citrates as liquid surfactant a) for producing cosmetic compositions, preference is given to pH regulators. For cosmetic compositions the pH established in this case is preferably a pH range of 4-8.

Examples of further customary cosmetic additives D) are emulsifiers, pearlescent waxes, stabilizers, salt, thickeners, consistency agents, self-tanning agents, pigments, antioxidants, anti-dandruff agents, film formers, swelling agents, insect repellents, active deodorant and antiperspirant ingredients, and active biogenic ingredients. These active biogenic ingredients are, in particular, tocopherol, tocopherol acetate, tocopherol palmitate, deoxyribonucleic acid, coenzyme Q10, ascorbic acid, retinol derivatives and retinyl derivatives, bisabolol, allantoin, phytantriol, panthenol, AHA acids, amino acids, ceramides, essential oils, hyaluronic acid, creatine, protein hydrolysates, plant extracts, peptides, and vitamin complexes.

For use as a cleansing gel or as a paste and ointment, preference is given to the presence as cosmetic additives D) of consistency agents and thickeners. Consistency agents contemplated include primarily fatty alcohols or hydroxy-fatty alcohols having 12 to 22 and preferably 16 to 18 carbon atoms, and also partial glycerides, fatty acids, or hydroxy-fatty acids. Preferred is a combination of these substances with alkyloligoglucosides and/or fatty acid N-methylglucamides of the same chain length and/or polyglycerol poly-12-hydroxystearates. Examples of suitable thickeners are Aerosil grades (hydrophilic silicas), polysaccharides, especially xanthan gum, guar guar, agar agar, alginates, and Tyloses, carboxymethylcellulose and hydroxyethyl- and hydroxypropylcellulose, and additionally relatively high molecular mass polyethylene glycol monoesters and diesters of fatty acids, polyacrylates and hydrophobically modified polyacrylates, polyacrylamides, polymers, polyvinyl alcohol, and polyvinylpyrrolidone. Also having proven particularly effective are bentonites, comprising a mixture of cyclopentasiloxane, disteardimonium hectorite, and propylene carbonate. Additionally contemplated are surfactants, such as, for example, ethoxylated fatty acid glycerides, esters of fatty acids with polyols such as pentaerythritol or trimethylolpropane, for example, fatty alcohol ethoxylates with narrowed homolog distribution, or alkyloligoglucosides, and also electrolytes such as sodium chloride and ammonium chloride.

If preservatives are used in accordance with the invention as additive D), they are preferably selected from the group consisting of benzoic acid and its salts, citric acid and its salts, phenoxyethanol, benzyl alcohol, alkyl parabens, preferably ethyl, methyl, and propyl paraben. Examples of further suitable preservatives include formaldehyde solution, pentanediol, or sorbic acid, and also the silver complexes known under the Surfacine® designation, and the further classes of substance listed in appendix 6, parts A and B of the Cosmetics Regulation.

Also advantageous as cosmetic additives D) are cationic compounds which give the skin a pleasant smooth sensation and softness. Included especially suitably are quaternary ammonium compounds or a cationic polymer. Quaternary ammonium compounds here mean, in particular, quaternized fatty acid triethanolamine ester salts. Also suitable, however, are alkylammonium halides. Examples of suitable cationic polymers are cationic cellulose derivatives, such as, for example, quaternized hydroxyethylcellulose, cationic starch, copolymers of diallylammonium salts and acrylamides, quaternized vinylpyrrolidone/vinylimidazole polymers, such as Luviquat (BASF), for example, condensation products of polyglycols and amines, quaternized collagen polypeptides, such as Lauryldimonium hydroxypropyl hydrolyzed collagen (Lamequat), for example, quaternized wheat polypeptides, polyethyleneimine, cationic silicone polymers, such as amidomethicones, copolymers of adipic acid and dimethylaminohydroxypropyldiethylenetriamine (Cartaretins), copolymers of acrylic acid with dimethyldiallylammonium chloride (Merquat 550), polyaminopolyamides, as described in FR-A 2252840, for example, and also crosslinked water-soluble polymers thereof, cationic chitin derivatives, such as quaternized chitosan, for example, optionally in microcrystalline distribution, condensation products of dihaloalkyls, such as dibromobutane, for example, with bisdialkylamines, such as bisdimethylamino-1,3-propane, for example, cationic guar gum, and quaternized ammonium salt polymers, such as, for example, Mirapol A-15, Mirapol AD-1, and Mirapol AZ-1 from Miranol.

Other preferred cationic polymers are selected from the group of the homopolymers or copolymers of ester derivatives or amide derivatives of acrylic or methacrylic acid (e.g. INCI: Polyquaternium-7, or PQ-7), homopolymers of methacryloylethyltrimethylammonium chloride (INCI: Polyquaternium-37, or PQ-37), quaternary copolymers of hydroxyethylcellulose and diallyldimethylammonium chloride (INCI: Polyquaternium-4, or PQ-4), polymeric quaternized ammonium salts of hydroxyethylcellulose which have been modified with a trimethylammonium-substituted epoxide (INCI: Polyquaternium-10, or PQ-10), depolymerized guar gum derivatives which have been quaternized (INCI: Guar Hydroxypropyl Trimonium Chloride), or quaternized guar derivatives and quaternary copolymers of hydroxyethylcellulose and diallyldemethylammonium chloride. In one preferred embodiment the cationic polymer is selected from the group consisting of Polyquaternium-7, Polyquaternium-10, and cationic guar derivatives. With advantage it is possible, moreover, to use cationic polymers in accordance with the teaching of EP 1 767 554 A1, these polymers being sold by the applicant company under the brand name Polyquart Pro. The cosmetic compositions of the invention preferably comprise 0.05 to 2 wt % of these cationic polymers.

In accordance with one especially suitable embodiment of the present invention, the cosmetic compositions comprise
A) 5.0 to 15 wt % of oil concentrates of the invention
B) 5.0 to 15 wt % of fatty alcohol ethersulfate
C) 2 to 10 wt % of cocoamidopropyl betaine
D) 0.5 to 5 wt % of cosmetic additives selected from the group of the preservatives and thickeners, preferably mixtures of ethoxylated and/or propoxylated polyol esters of trimethylolpropane and ethoxylated fatty alcohols as thickeners, and also
E) water ad 100 wt %.

By incorporating the oil concentrate as component A) into the cosmetic compositions of the invention, success is achieved in the transparent to slightly turbid incorporation of relatively large amounts of oily substances which induce an outstanding skin sensation while nevertheless allowing an acceptable quantity of foam. As a result, they differ advantageously from other cosmetic compositions which comprise oils. Hence the use of the oil concentrate is essential to the advantageous properties of the cosmetic compositions of the invention.

### EXAMPLES

### Example A

### Oil Concentrates

All of the components (i.e., components a), b) c1), and optionally c2) were homogenized at room temperature (23° C.) with moderate mechanical input. The resulting oil concentrates were evaluated visually, and their solubility in water was tested.

The inventive examples (Inv) all show that liquid anionic surfactants of type a) in blends with the nonionic surfactants (b) and with a polar oil (c1), optionally in a mixture with (c2), always produce clear, liquid oil concentrates which are readily dispersible in water to form a fine, stable emulsion.

In comparison to these (Comp), oil concentrates without anionic liquid surfactants (a) (comparative example A17) or without nonionic surfactants (b) (i.e., comparative example A18), and also with other liquid anionic surfactants of the lactate type (see comparative example A19) exhibit precipitation after several days.

| Example | 1 | 2 | 3 | 4 | 5 | 6 | 7 | 8 | 9 | |
|---|---|---|---|---|---|---|---|---|---|---|
| I nventive (Inv)/Comparative | | | | | | | | | | |
| (Corn) | Inv | Inv | Inv | Inv | Inv | Inv | Inv | Inv | Inv | |
| IngredientINC! | Corn | Wt. | Wt. | Wt. | Wt. | Wt. | Wt. | Wt. | Wt. | Wt. |
| Po- | +%+ | +%+ | +%+ | +%+ | +%+ | +%+ | +%+ | +%+ | +%+ | |
| nent | | | | | | | | | | |
| De- | Polyglycery1-2 Dipolyhydroxys- | 20 | 20 | 20 | 20 | 20 | 20 | 20 | 20 | 20 |
| hymuls | tearate | | | | | | | | | |
| PGPH?+0 | | | | | | | | | | |
| 3 | Arlypon | PEG/PPG-120/10 Trime- | d) | | | | | | | |
| TT?+0 | thylolpropane | | | | | | | | | |
| Trioleate (and) Laureth-2 | | | | | | | | | | |
| Ong. | Mc.) P | | | | | | | | | |
| 5 | Soybean | Glycine Soja (Soybean) Oil | c1) | 25 | | | | | | |
| Oil | . | | | | | | | | | |
| 7 | Rape- | Brassica Campestris (Rape- | c1) | 25 | 50 | 25 | | | | |
| seed oil | seed) Seed Oil | | | | | | | | | |
| (Rapso) | | | | | | | | | | |

| Example | 10 | 11 | 12 | 13 | 14 | 15 | 16 | 17 | 18 | 19 | | |
|---|---|---|---|---|---|---|---|---|---|---|---|---|
| Inventive | | | | | | | | | | | | |
| (Inv)/Comparative (Corn) | Inv | Inv | Inv | Inv | Inv | Inv | Inv | Corn | Corn | Corn | | |
| Ingredient | INCI | Corn | Wt. | Wt. | Wt. | Wt. | Wt. | Wt. | Wt. | Wt. | Wt. | Wt. |
| Po- | +%+ | +%+ | +%+ | +%+ | +%+ | +%+ | +%+ | +%+ | +%+ | +%+ | | |
| nent | | | | | | | | | | | | |
| 1 | Dehymuls | Polyglyceryl-2 Dipolyhy- | b) | 20 | 20 | 20 | 20 | 20 | 16 | 50 | 0 | 20 |
| PGPH?+0 | droxystearate | | | | | | | | | | | |
| n 8 | | | | | | | | | | | | |
| 3 | Lameform | Polyglyceryl-3 Diisos- | b) | 20 | | | | | | | | |
| TGI | tearate | | | | | | | | | | | |
| d rd | | | | | | | | | | | | |
| 5 | Luvitol | Hydrogenated Polyisobu- | c2) | 20 | 20 | 25 | 25 | 25 | 20 | | | |
| Lite?+0 | tene | | | | | | | | | | | |

Assignment of “clear”, “turbid”, and “liquid” was made as a result of visual assessment. For the evaluation of “soluble”, a 3 wt % strength aqueous solution in water was prepared, and the solubility was ascertained visually.

### Example B

### Shower Products

Water 1, sodium benzoate, and also Texapon N 70® and Dehyton PK45® are homogenized at room temperature, the oil concentrate, the Arlypon TT® thickener, and the water 2 are stirred into the mixture in succession at room temperature (23° C.), and subsequently the pH of the composition is adjusted. Inventive shower products 20 to 36 exhibit good storage stability, meaning that there are no instances of precipitation of the oil c1 and optionally c2 from the oil concentrates. Comparative experiment 37 shows that on direct introduction of the oil, without preformulation via an oil concentrate, the storage stability is poor and the oil already undergoes separation even at room temperature.

| Example B | Inv | Inv | Inv | Inv | Inv | Inv | |
|---|---|---|---|---|---|---|---|
| Shower product | 20 | 21 | 22 | 23 | 24 | 25 | |
| INCI | Wt% | Wt% | Wt% | Wt% | Wt% | Wt% | |
| Water 1 | Aqua | 10.4 | 10.4 | 10.4 | 10.4 | 10.4 | 10.4 |
| Sodium benzoate | SODIUM BENZOATE | 0.5 | 0.5 | 0.5 | 0.5 | 0.5 | 0.5 |
| Texapon NSO? (27 | Sodium Laureth Sulfate | 44 | 44 | 44 | 44 | 44 | 44 |
| wt% ) | | | | | | | |
| Plantacare 818 UP? Coco-Glucoside | | | | | | | |
| (52 wt%) | | | | | | | |
| Dehyton PK 45+169 | Cocamidopropyl Betaine | 7.8 | 7.8 | 7.8 | 7.8 | 7.8 | 7.8 |
| (38.5 wt%) | | | | | | | |
| Oil concentrate of | 10 | | | | | | |
| example 1 | | | | | | | |
| Oil concentrate of | 10 | | | | | | |
| example 3 | | | | | | | |
| Oil concentrate of | 10 | | | | | | |
| example 6 | | | | | | | |
| Oil concentrate of | 10 | | | | | | |
| example 7 | | | | | | | |
| Oil concentrate of | 10 | | | | | | |
| example 8 | | | | | | | |
| Oil concentrate of | 10 | | | | | | |
| example 9 | | | | | | | |
| Oil concentrate of | | | | | | | |
| example 10 | | | | | | | |
| Oil concentrate of | | | | | | | |
| example 11 | | | | | | | |
| Oil concentrate of | | | | | | | |
| example 12 | | | | | | | |
| Oil concentrate of | | | | | | | |
| example 13 | | | | | | | |
| Oil concentrate of | | | | | | | |
| example 14 | | | | | | | |

| Example B | Inv | Inv | Inv | Inv | Inv | Inv | |
|---|---|---|---|---|---|---|---|
| Shower product | 26 | 27 | 28 | 29 | 30 | 31 | |
| INCI | Wt% | Wt% | Wt% | Wt% | Wt% | Wt% | |
| Water 1 | Aqua | 9.2 | 9.2 | 10.4 | 10.4 | 10.4 | 11.4 |
| Sodium benzoate | SODIUM BENZOATE | 0.5 | 0.5 | 0.5 | 0.5 | 0.5 | 0.5 |
| Texapon NSO? (27 | Sodium Laureth Sulfate | 44 | 44 | 44 | 44 | 44 | 44 |
| wt% ) | | | | | | | |
| Plantacare 818 UP? Coco-Glucoside | 2.9 | | | | | | |
| (52 wt%) | | | | | | | |
| Dehyton PK 45+169 | Cocamidopropyl Betaine | 7.8 | 7.8 | 7.8 | 7.8 | 7.8 | 3.9 |
| (38.5 wt%) | | | | | | | |
| Oil concentrate of | | | | | | | |
| example 1 | | | | | | | |
| Oil concentrate of | | | | | | | |
| example 3 | | | | | | | |
| Oil concentrate of | | | | | | | |
| example 6 | | | | | | | |

Example B

| Shower product | 32 | 33 | 34 | 35 | 36 | 37 | |
|---|---|---|---|---|---|---|---|
| INCI | Wt% | Wt% | Wt% | Wt% | Wt% | Wt% | |
| Water 1 | Aqua | 11.4 | 11.4 | 11.4 | 11.11 | 9 | 14 |
| Sodium benzoate | SODIUM BENZOATE | 0.5 | 0.5 | 0.5 | 0.5 | 0.5 | 0.5 |
| Texapon NSO? (27 | Sodium Laureth Sulfate | 44 | 44 | 44 | 44 | 44 | 44 |
| wt% ) | | | | | | | |
| Plantacare 818 UP? Coco-Glucoside | 2.9 | 2.9 | 2.9 | 2.9 | | | |
| (52 wt%) | | | | | | | |
| Dehyton PK 45+169 | Cocamidopropyl Betaine | 3.9 | 3.9 | 3.9 | 3.9 | 7.8 | 7.8 |
| (38.5 wt% ) | | | | | | | |
| Oil concentrate of | | | | | | | |
| example 1 | | | | | | | |
| Oil concentrate of | | | | | | | |
| example 3 | | | | | | | |
| Oil concentrate of | | | | | | | |
| example 6 | | | | | | | |
| Oil concentrate of | | | | | | | |
| example 7 | | | | | | | |
| Oil concentrate of | 10 | | | | | | |
| example 8 | | | | | | | |
| Oil concentrate of | | | | | | | |
| example 9 | | | | | | | |
| Oil concentrate of | | | | | | | |
| example 10 | | | | | | | |
| Oil concentrate of | | | | | | | |
| example 11 | | | | | | | |
| Oil concentrate of | 10 | | | | | | |
| example 12 | | | | | | | |
| Oil concentrate of | 10 | | | | | | |
| example 13 | | | | | | | |
| Oil concentrate of | 10 | | | | | | |
| example 14 | | | | | | | |
| Oil concentrate of | 10 | | | | | | |
| example 16 | | | | | | | |
| Oil: | | | | | | | |
| Soybean oil | 2.5 | | | | | | |
| Liquid paraffin | 2.5 | | | | | | |

The viscosity was measured by Brookfield at RT (23° C.).

1. An oil concentrate for cosmetic compositions, comprising
(a) at least one anionic surfactant which is liquid at room temperature and is selected from the group of the alk(en)yl polyglycol ether citrates, and
(b) at least one nonionic surfactant, and
(c) an oil component with
(c1) a polar oil having a polarity of between 5 and 30 mN/m and optionally
(c2) an apolar oil having a polarity of above 35 mN/m, the (c1): (c1+c2) mixing ratio being in the range from 0.1 to 1,
(d) and also, optionally, thickeners.

2. The oil concentrate according to claim 1, comprising (a) as anionic surfactant alk(en)yl polyglycol ether citrates of the formula (I) and/or salts thereof 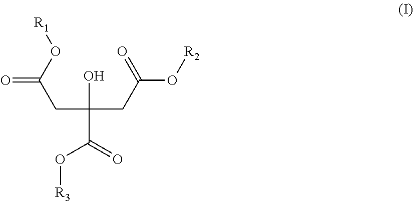
in which
R₁, R₂, and R₃ independently of one another are hydrogen or the radical of the formula
R₄(OCH₂CHR₅)ₙ (II)
in which
R₄ is a linear or branched alkyl and/or alkenyl radical having 6 to 22 carbon atoms,
R₅ is hydrogen or methyl radical, and
n is a number from 1 to 20, subject to the condition that at least one of the radicals, R₁, R₂, or R₃, is other than hydrogen.

3. The oil concentrate according to claim 1, comprising (a) as anionic surfactant alk(en)yl polyglycol ether citrates of the formula (I) in which R₁, R₂, or R₃ is the radical of the formula (II) and R₄ is a linear alkyl radical having 12 to 14 carbon atoms, R₅ is hydrogen, and n is a number from 5 to 10.

4. The oil concentrate according to claim 1, comprising as nonionic surfactant (b) esters of polyglycerols.

5. The oil concentrate according to claim 1, comprising polar oils (c1) having a polarity of between 15 and 28 mN/m.

6. The oil concentrate according to claim 1, comprising polar oils (c1) selected from the group consisting of olive oil, soybean oil, sunflower oil, almond oil, argan oil and avocado oil.

7. The oil concentrate according to claim 1, further comprising (c2) apolar oils having a polarity of between 35 and 60 mN/m.

8. The oil concentrate according to claim 1, comprising liquid paraffins and/or hydrogenated polyisobutenes as additional apolar oils (c2).

9. The oil concentrate according to claim 1, wherein the polar oils:apolar oils (c 1):(c 1+c2) mixing ratio is in the range from 0.3 to 0.7, preferably from 0.4 to 0.6, and more particularly from 0.45 to 0.55.

10. The oil concentrate according to claim 1, comprising as thickeners (d) mixtures of ethoxylated and/or propoxylated polyol esters of trimethylolpropane and ethoxylated fatty alcohols.

11. The oil concentrate according to claim 1, comprising
(a) alk(en)yl polyglycol ether citrates in amounts of 20 to 40 wt %, and
(b) at least one nonionic surfactant in amounts of 10 to 30 wt %, and also the
(c) oil component in amounts of 30 to 60 wt %, and also, optionally,
(d) thickeners in amounts of 0 to 15 wt %.

12. A cosmetic composition comprising
A) oil concentrate according to claim 1 and
B) anionic surfactants and
C) further, co-surfactants different from (B), and
D) cosmetic additives, and
E) water.

13. The cosmetic composition according to claim 12, comprising
A) 0.5 to 20 wt % of oil concentrate comprising
(a) at least one anionic surfactant which is liquid at room temperature and is selected from the group of the alk(en)yl polyglycol ether citrates, and
(b) at least one nonionic surfactant, and
(c) an oil component with
(c1) a polar oil having a polarity of between 5 and 30 mN/m and optionally
(c2) an apolar oil having a polarity of above 35 mN/m,
the (c1):(c1+c2) mixing ratio being in the range from 0.1 to 1,
(d) and also, optionally, thickeners,
B) 5.0 to 20 wt % of anionic surfactants,
C) 1 to 15 wt % of further, co-surfactants, different from (B),
D) 0.1 to 10 wt % of cosmetic additives, and
E) water ad 100 wt %.

14. The cosmetic composition according to claim 12, comprising
A) 5.0 to 15 wt % of oil concentrate according to claim 1 comprising
(a) at least one anionic surfactant which is liquid at room temperature and is selected from the group of the alk(en)yl polyglycol ether citrates, and
(b) at least one nonionic surfactant, and
(c) an oil component with
(c1) a polar oil having a polarity of between 5 and 30 mN/m and optionally
(c2) an apolar oil having a polarity of above 35 mN/m, the (c1):(c1+c2) mixing ratio being in the range from 0.1 to 1,
(d) and also, optionally, thickeners,
B) 5.0 to 20 wt % of anionic surfactants selected from the group consisting of fatty alcohol ether sulfate, monoglyceride ether sulfate, mono- and dialkylsulfosuccinate, ethercarboxylic acids and/or salts thereof, acylglutamates, and alkyloligoglucoside sulfates
C) 1 to 10 wt % of amphoteric and/or cationic surfactants
D) 0.5 to 5.0 wt % of cosmetic additives, and
E) water ad 100 wt %.

15. A method for producing oil-containing cosmetic compositions, which comprises stirring together oil concentrate according to claim 1 with
B) anionic surfactants and
C) nonionic surfactants and
D) cosmetic additives, and
E) water
in a cold operation.

16. The oil concentrate according to claim 1 comprising an nonionic surfactant ester of polyglycerols with poly-12-hydroxystearic acid.

17. The oil concentrate according to claim 1 where the polar oils:apolar oils (c1):(c1+c2) mixing ratio is in the range of 0.4 to 0.6.

18. The oil concentrate according to claim 11 comprising 25 to 35 wt % of the alk(en)yl polyglycol ether citrates.

19. The oil concentrate according to claim 11 comprising 15 to 25 wt % of the at least one nonionic surfactant.

20. The oil concentrate according to claim 11 comprising 40 to 55 wt % of the oil component.
